(19) United States
(12) Patent Application Publication
(10) Pub. No.: US 2022/0168560 A1
(43) Pub. Date: Jun. 2, 2022

(54) GARMENT PAIRED WITH A LEG CONTRACTION IMPULSE DEVICE THAT IS TRIGGERED BY WALKING

(71) Applicant: SYSTAGENIX WOUND MANAGEMENT, LIMITED, West Sussex (GB)

(72) Inventors: Daniel IBBITSON, West Sussex (GB); Matthew RAMSEY, West Sussex (GB); Alexander WAITE, Keighley (GB); Daniel PARKER, West Sussex (GB)

(21) Appl. No.: 17/440,005

(22) PCT Filed: Apr. 1, 2020
(86) PCT No.: PCT/IB2020/053067

§ 371 Date: Sep. 16, 2021

Related U.S. Application Data
(60) Provisional application No. 62/835,722, filed on Apr. 18, 2019.

(51) Int. Cl. A61N 1/04; A61B 5/00; A61N 1/36

(52) U.S. Cl. CPC A61N 1/0484; A61B 5/6807; A61B 2562/0247; A61N 1/0452; A61N 1/36034; A61N 1/36003

(57) ABSTRACT

A garment for stimulating venous return includes a woven textile, an electrode, and a sensor. The woven textile includes calf muscle portion configured to be disposed proximate to a calf muscle and a foot portion configured to be disposed proximate to a foot. The electrode is operably coupled to the calf muscle portion of the woven textile. The sensor is disposed on the foot muscle portion of the woven textile and is communicatively coupled to the electrode. The sensor is configured to generate a control signal in response to user movement. The electrode is configured to activate in response to the control signal.

### CROSS-REFERENCE TO RELATED APPLICATIONS

This application claims the benefit of priority to U.S. Provisional Application No. 62/835,722, filed on Apr. 18, 2019, which is incorporated herein by reference in its entirety.

### BACKGROUND

The present disclosure relates generally to the treatment of poor venous return. More specifically, the present disclosure relates to the use of an impulse device paired with a compression garment to improve venous return.

The term venous return refers to blood return from a person's outer extremities. Many people suffer from the condition of poor venous return, specifically in their lower extremities such as in their legs. Poor venous return can result from numerous issues including faulty blood flow return valves that prevent blood from leaving the leg. Left untreated, poor venous return can increase the risk of developing conditions such as venous leg ulcers, which are linked to even more serious conditions such as Lipodermatosclerosis, Oedema, and scarring.

A conventional treatment for poor venous return involves the application of compression socks or bandaging, which promote blood return by applying pressure along the lower part of the leg (e.g., along a calf portion of the leg and a foot). However, identifying the correct pressure to apply to the leg can be difficult, and even small amounts of pressure can result in user discomfort. Accordingly, devices and methods are desired that improve venous return.

### SUMMARY OF THE INVENTION

One implementation of the present disclosure is a garment for stimulating venous return. The garment includes a woven textile, an electrode, and a sensor. The woven textile includes a calf muscle portion configured to be disposed proximate to a calf muscle and a foot portion configured to be disposed proximate to a foot. The electrode is operably coupled to the calf muscle portion of the woven textile. The sensor is disposed on the foot muscle portion of the woven textile and is communicatively coupled to the electrode. The sensor is configured to generate a control signal in response to user movement. The electrode is configured to activate in response to the control signal.

In some embodiments, the foot portion of the woven textile includes a heel portion configured to be disposed proximate to a heel of the foot. The sensor may be operably coupled to the heel portion. The sensor may include a pressure sensor configured to generate the control signal in response to a force applied to the heel portion. The pressure sensor may be configured to deactivate the control signal in response to the force being removed from the heel portion.

In any of the above embodiments, the calf muscle portion of the woven textile comprises a common perineal nerve portion configured to be disposed proximate to a common perineal nerve. The electrode may be disposed proximate to the common perineal nerve portion.

In any of the above embodiments, the electrode may be configured to operate in either an activated state in which the electrode is configured to stimulate the calf muscle or a deactivated state in which the electrode is not configured to stimulate the calf muscle. The electrode may be configured to pulsate continuously between the activated state and the deactivated state after a predetermined period of time has elapsed during which the electrode is in the deactivated state.

In any of the above embodiments, the garment may further include a battery and a kinetic charger coupled thereto. The kinetic charger may be configured to supply current to the battery. In some embodiments, the garment may include conductive fibers woven into the woven textile and electrically coupled to at least one of the electrode and the sensor.

Another implementation is an assembly for stimulating venous return. The assembly includes a sock including a hollow sleeve. The hollow sleeve includes a calf muscle portion configured to receive a calf muscle and a foot portion configured to receive a foot. The hollow sleeve is configured to apply a pressure to the calf muscle and the foot. The assembly also includes an electrode and a sensor. The electrode is disposed in the hollow sleeve proximate to the calf muscle portion. The sensor is disposed in the hollow sleeve proximate to the foot portion. The sensor is configured to generate a control signal in response to user movement. The electrode is configured to activate the electrode in response to the control signal.

In any of the above embodiments, the assembly may include a step-counter disposed in the hollow sleeve and configured to record the movement. In some embodiments, the step-counter may be configured to record a number of control signals generated by the sensor.

In any of the above embodiments, the sock may include a long stretch material. In some embodiments, the electrode and the sensor may be woven into the sock.

Another implementation is a method of making a compression garment for stimulating venous return. The method includes providing a woven textile including a calf muscle portion configured to be disposed proximate to a calf muscle, and a foot portion configured to be disposed proximate to a foot. The method further includes providing an electrode. The electrode is configured to activate and stimulate the calf muscle in response to a control signal. The method also includes providing a sensor configured to generate a control signal in response to an applied force. The method further includes integrating the electrode into the calf muscle portion of the woven textile. The method further includes integrating the sensor into the foot portion of the woven textile. The method also includes electrically coupling the sensor to the electrode.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a rear perspective view of a compression garment and leg contraction impulse device, according to an exemplary embodiment.

FIG. 2 is a rear view of a leg contraction impulse device showing the location of an electrode for the device, according to an exemplary embodiment.

FIG. 3 is a side perspective view of a leg contraction impulse device showing the location of an electrode for the device, according to an exemplary embodiment.

FIG. 4 is a schematic diagram of an electrical circuit for a leg contraction impulse device, according to an exemplary embodiment.

FIG. 5 is an operational schematic for a leg contraction impulse device, according to an exemplary embodiment.

FIG. 6 is a block diagram showing a method of manufacture for a compression sock and leg contraction impulse device, according to an exemplary embodiment.

### DETAILED DESCRIPTION

### Overview

Referring generally to the FIGURES, a garment for stimulating venous return is provided, according to various exemplary embodiments. The garment includes a sensor in a foot portion of the garment. The sensor is configured to cause an electrode to activate in response to a person's movement. Once activated, the electrode is configured to cause a calf muscle to contract (e.g., to produce an electrical impulse or otherwise cause the calf muscle to contract). The position of the sensor coordinates the electrical impulse with the natural contraction of the leg muscle due to movement, which can, advantageously, reduce any pain that might be associated with the electrical impulse. The electrical impulse will cause the calf muscle to contract even more than would normally be observed (e.g., than would normally be observed when walking, running, etc.), which will improve venous return.

The garment may be a sock provided as part of an assembly for stimulating venous return. The sock may include a hollow sleeve including a calf muscle portion configured to receive a calf muscle and a foot portion configured to receive a foot. The sock may be a compression sock made from long stretch material that applies a pressure to the leg so as to further promote blood return from the leg. The electrode may be disposed in the hollow sleeve proximate to the calf muscle portion. The sensor may be disposed in the hollow sleeve proximate to a heel portion of the sock. Among other benefits, positioning the electrode in the heel portion of the sock substantially coordinates the stimulatory effect associated with the electrical impulse with the natural contraction of the calf muscle due to regular movement.

A method of assembly for the garment includes providing the woven textile, providing the electrode, and providing the sensor. The method additionally includes integrating the electrode into the calf muscle portion of the woven textile, integrating the sensor into the foot portion of the woven textile, and coupling the sensor to the electrode. These and other features and advantages of the garment are described in detail below.

### Garment Construction

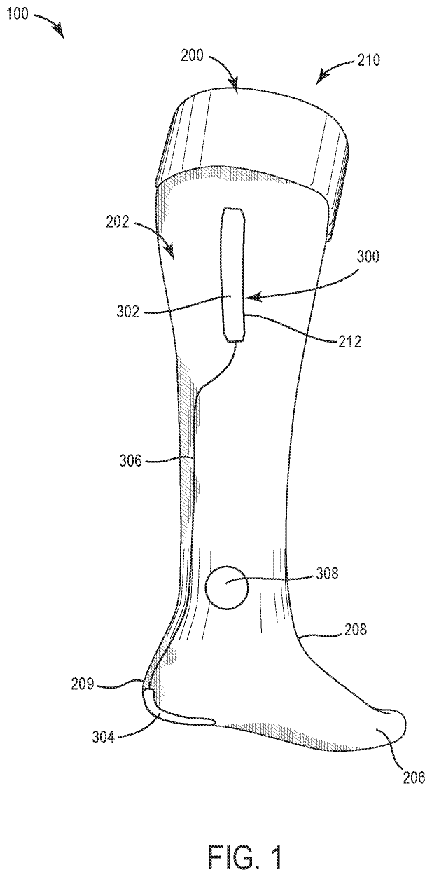

Referring now to FIG. 1, a garment 100 for stimulating venous return is shown, according to an exemplary embodiment. The garment 100 is configured to receive a portion of a person's leg, including a calf muscle and a foot. As shown in FIG. 1, the garment 100 includes a woven textile, shown as sock 200. The sock 200 includes a hollow sleeve 202 including a calf muscle portion 204 configured to receive the calf muscle and a foot portion 206 configured to receive the foot. In an exemplary embodiment, the sock 200 is a compression sock configured to apply a pressure to the leg. In FIG. 1, the sock 200 is made from a long stretch material so as to conform with the calf muscle as it contracts during movement. Among other benefits, using a long stretch material may increase the effectiveness of venous return from the leg as compared with a short stretch material.

As shown in FIG. 1, the sock 200 includes an electrical impulse device 300. The impulse device 300 is configured to provide an electrical impulse to the calf muscle, and to coordinate the impulse with a person's movement (e.g., in response to a force applied to the leg, etc.). The impulse device 300 includes an electrode 302 disposed in the calf muscle portion 204 of the hollow sleeve 202. The electrode 302 is coupled to the hollow sleeve 202 and positioned in between the hollow sleeve 202 and the calf muscle. The electrode 302 is configured to contact the calf muscle so as to transmit an electrical impulse to the calf muscle. The electrode 302 is configured to operate in either an activated state in which the electrode 302 is configured to stimulate the calf muscle or a deactivated state in which the electrode is not configured to stimulate the calf muscle. In one embodiment, the electrode 302 is disposed at a common perineal nerve portion of the calf muscle portion 204, which can, advantageously, maximize an amount of contraction of the calf muscle resulting from the electrical impulse.

As shown in FIG. 1, the electrical impulse device 300 additionally includes a sensor 304 disposed in the hollow sleeve 202 proximate to the foot portion 206. The sensor 304 is configured to generate a control signal in response to movement of the foot portion 206. In some embodiments, the sensor 304 is configured to generate the control signal in response to a pressure applied to the foot portion 206. As shown in FIG. 1, the sensor 304 is operably coupled to the electrode 302. The sensor 304 may be electrically coupled to the electrode using a conductive member 306. The conductive member 306 may be coupled to an outer surface 208 of the hollow sleeve 202. Alternatively, the conductive member 306 may be a conductive fiber woven into the sock 200.

As shown in FIG. 1, the conductive member 306 extends from the foot portion 206 of the hollow sleeve 202 toward the calf portion 204 of the hollow sleeve 202, between the sensor 304 and the electrode 302. The sensor 304 is configured to transmit the control signal to the electrode 302 in response to a user's movement. The electrode 302 is configured to activate in response to the control signal, thereby coordinating the application of the electrical impulse with the natural contraction of the calf muscle that occurs from user movement. In some embodiments, the sensor 304 is disposed proximate to a heel portion 209 of the foot portion 206 so as to coordinate the application of the impulse with the natural contraction of the calf muscle that results from taking a step forward (e.g., the contraction associated with contact between the user's foot and a surface as the user lowers their foot onto the surface, etc.).

The electrical impulse device 300 may additionally include a power source 308. As shown in FIG. 1, the power source 308 is a battery that is electrically coupled to at least one of the electrode 202 and the sensor 304. The electrical impulse device 300 may include a kinetic charging device configured to supply current to the battery in response to movement of the leg. The electrical impulse device 300 may also include a movement tracking device such as a step-counter configured to log the person's movements over time (e.g., to monitor and log each time the sensor 304 generates the control signal, to monitor and log each time the electrode 302 is activated, etc.). The electrical impulse device 300 may be configured to activate the electrode 302 after a period of time has elapsed during which the electrode 302 is inactive (e.g., after a period of time has elapsed where the step-counter has not observed any user movement, after a period of time has elapsed during which the amount of user movement was limited, etc.). Activating the electrode 302 during periods of inactivity can, advantageously, reduce the likelihood that venous return is inhibited by sedentary behavior (e.g., while the person is at rest, while the person is sitting down, etc.). In an exemplary embodiment, the electrode 302 is configured to pulsate at regular intervals after a period of time has elapsed during which the electrode 302 is inactive.

According to an exemplary embodiment, the electrical impulse device 300 is at least partially detachable (e.g., removable) from the sock 200. In FIG. 1, all of the electrical components, including the electrode 302, the sensor 304, and the power source 308 may be removed from the sock 200. Among other benefits, using detachable components reduces replacement costs for the sock 200, which may be replaced separately from the other components. Using removable components also allows the user to maintain (e.g., wash, etc.) the sock 200, which may degrade or become dirty after prolonged periods of use.

### Woven Textile

An exemplary embodiment of a woven textile for the garment 100 is shown in FIG. 1. The woven textile, shown as sock 200, includes a hollow sleeve 202 configured to receive a lower part of a leg. The sock 200 may be a compression sock configured to apply pressure to the leg to promote venous return. The hollow sleeve 202 may be made from a variety of materials including nylon, cotton, spandex, and other compliant materials. The hollow sleeve 202 may be machine washable. In FIG. 1, the hollow sleeve 202 is made from a long stretch material having a high elasticity. Among other benefits, using a long stretch material enables freedom-of movement of the leg within the sock 200 and may reduce pain during ambulation (e.g., during periods when the person is walking, running, etc.). Using a long stretch material also provides space for the electrical components of the impulse device 300 to fit in-between the sock 200 and the leg.

Although there are many benefits associated with using a long stretch material, other materials have also been considered, and may be used without significantly impacting the effectiveness of the treatment. For example, the hollow sleeve 202 may also be made from materials with limited stretch (e.g., short stretch materials) and/or higher working pressure as compared to a long stretch material. In some implementations, the hollow sleeve 202 may be made from a multilayer material including some combination of long and short stretch materials.

As shown in FIG. 1, the hollow sleeve 202 defines a cavity 210 configured to receive a portion of the leg. The hollow sleeve 202 is configured to substantially surround and contain the leg. The hollow sleeve 202 includes a calf muscle portion 204 configured to be disposed proximate to a calf muscle, and a foot portion 206 configured to be disposed proximate to a foot. The foot portion 206 includes a heel portion 209 configured to be disposed proximate to a heel of the foot. As shown in FIG. 1, the calf muscle portion 204 of the hollow sleeve 202 extends upward from the foot portion 206. In the embodiment of FIG. 1, the calf muscle portion 204 includes a common perineal nerve portion 212 configured to be disposed proximate to a common perineal nerve on the upper part of the leg. A length of either portion 204, 206 of the sock 200 may vary depending on the person's size and leg compression requirements.

In some embodiments, the sock 200 may include a conductive member 306 configured to operably couple various electrical components including the electrode 302, the sensor 304, etc. to to the sock 200. As shown in FIG. 1, the conductive member 306 may be coupled (e.g., adhered, strapped, etc.) to an outer surface 208 of the hollow sleeve 202. Alternatively, the conductive member 306 may be woven or otherwise embedded into one or more layers of the hollow sleeve 202. The conductive member 306 may include solid wire or conductive fibers that extend along a length of the hollow sleeve 202 (e.g., a length of the hollow sleeve 202 substantially parallel to a primary axis of the hollow sleeve 202, etc.).

In some embodiments, the sock 200 may include pockets or slots configured to receive and retain one or more electrical components. These pockets can, advantageously, prevent the electrical components from being inadvertently removed from the hollow sleeve 202 during normal use (e.g., from becoming dislodged from the hollow sleeve 202 during normal movement of the leg, etc.). The sock 200 may additionally include connectors (e.g., electrical connectors) configured to communicatively couple (e.g., electrically connect) the electrical components to the conductive members 306 in the sock 200. Among other benefits, the connectors may enable a user to detach the electrical components from the sock 200 in order to wash the sock, or to replace damaged components individually rather than replacing the entire garment 100. The connectors may be one, or a combination of, of a variety of different connectors known to those of ordinary skill in the art.

### Electrical Impulse Device

Referring now to FIGS. 1-4, an electrical impulse device 300 is shown, according to an exemplary embodiment. The electrical impulse device 300 is configured to stimulate a calf muscle in response to user movement. As shown in FIGS. 1-4, the electrical impulse device 300 includes an electrode 302 configured to stimulate the calf muscle. As shown in FIG. 1, the electrode 302 is operably coupled to the calf muscle portion of the hollow sleeve 202. The electrode 302 is configured to be disposed between the hollow sleeve 202 and the calf muscle.

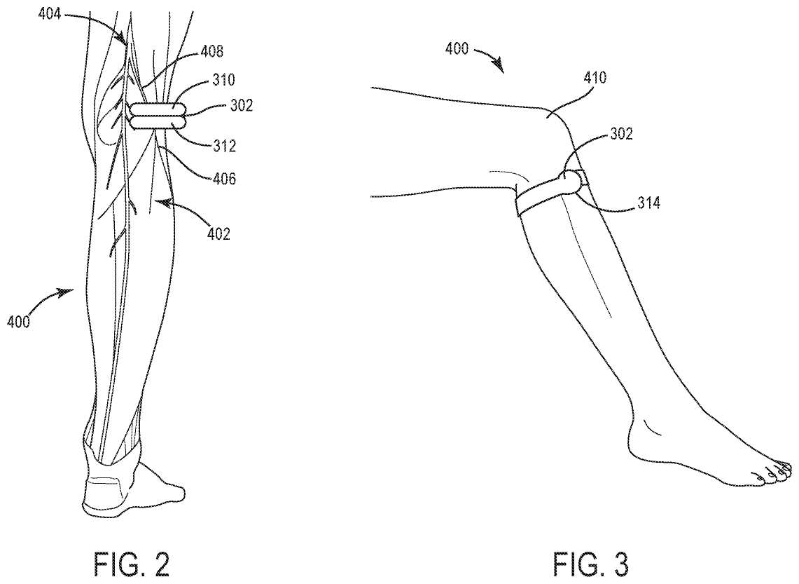

FIGS. 2-3 show the position of the electrode 302 relative to different portions of a lower leg, according to an exemplary embodiment. As shown in FIG. 2, the electrode 302 includes a positive terminal 310 and a negative terminal 312. The terminals 310, 312 are shown in contact with the skin of the leg 400. The terminals 310, 312 are engaged with the skin so as to deliver an electrical impulse to a calf muscle 402. FIG. 2 shows the position of the terminals 310, 312 in relation to the calf muscle 402 and also in relations to nerves 404 extending along a length of the leg (e.g., from a top side of the leg 400 to a bottom side of the leg 400). As shown in FIG. 2, both the positive terminal 310 and the negative terminal 312 are disposed proximate to an upper portion 406 of the calf muscle 402. According to an exemplary embodiment, the terminals 310, 312 are disposed proximate to a common peroneal nerve 408, which extends downwardly along an upper portion of the calf muscle 402. Among other benefits, positioning the terminals 310, 312 of the electrode 302 across (e.g., over, centrally with respect to, etc.) the common perineal nerve 408 reduces the amount of energy needed to induce a medically relevant amount of contraction of the calf muscle 402.

The exact point of application may differ between users depending on their leg size and/or whether any wounds exist that limit access to the leg. In some embodiments, the terminals 310, 312 may be positioned along a length of the calf muscle portion 204 of the hollow sleeve 202 (see also FIG. 1) so as to engage with a length of the calf muscle 402 parallel to the leg 400, etc.). In yet other embodiments, the terminals 310, 312 may be disposed at other positions along the calf muscle portion 204 or foot portion 206 to promote venous return.

Referring now to FIG. 3, an electrode 302 is shown engaged with a user's leg 400, according to an exemplary embodiment. As shown in FIG. 3, the electrode 302 is engaged with the skin of the leg 400 just to the right side of a kneecap 410 of the leg 400. The electrode 302 may be configured in a variety of different shapes and sizes. The electrode 302 may include a housing 314 configured to receive and support the terminals 310, 312. The housing 314 may facilitate placement of the terminals 310, 312 when the hollow sleeve 202 (see also FIG. 1) is placed over the leg 400. The housing 314 may also insulate the terminals 310, 312 both from one another and from the hollow sleeve 202, thereby preventing accidental shock or stimulation of other areas of the body. As shown in FIG. 3, the electrode 302 is substantially circular with an outer diameter that is approximately the same as a length of the terminals 310, 312. In other embodiments, the shape and/or size of the electrode 302 or housing 314 may be different.

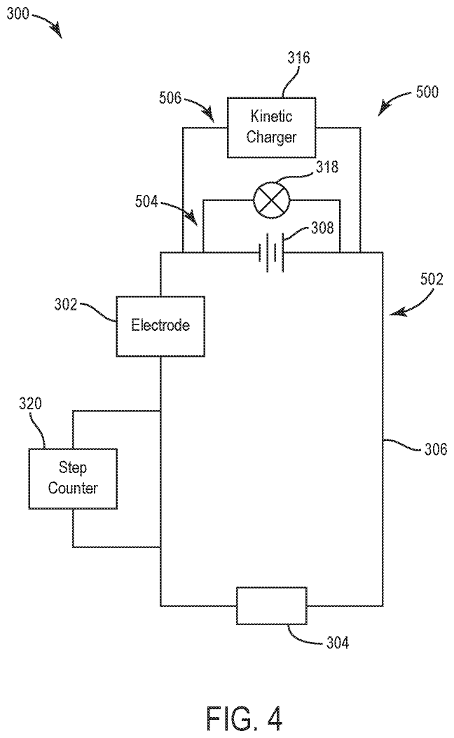

Referring now to FIG. 4, a schematic diagram of a circuit 500 for the electrical impulse device 300 is shown, according to an exemplary embodiment. The impulse device 300 includes a plurality of electrical components configured to control and power the electrode 302. In other embodiments, more or fewer electrical components may be included. As shown in FIG. 4, the impulse device 300 includes a power source 308 coupled (e.g., electrically connected) to the electrode 302. The power source 308 may include a battery such as a lithium-ion battery, or another compact or lightweight battery type. As shown in FIG. 1, the power source 308 is disposed in the hollow sleeve 202, just below the electrode 302. In other embodiments, the position of the power source 308 with respect to the hollow sleeve 202 may be different. The power source 308 may be disposed on an outer surface 208 of the hollow sleeve 202 for ease-of-access (e.g., so that sock 200 doesn't have to be removed from the leg to access the power source 308, etc.). In some embodiments, the power source 308 may be detachable (e.g., removable, etc.) from the hollow sleeve 202. Among other benefits, using a detachable power source 308 enables a user to quickly replace a power source 308 once discharged and/or remove the power source 308 to facilitate cleaning of the hollow sleeve 202.

As shown in FIG. 4, the electrical impulse device 300 includes a sensor 304 communicatively coupled (e.g., electrically connected) to the electrode 302. According to an exemplary embodiment, the sensor 304 is positioned within the hollow sleeve 202 so as to coordinate activation of the electrode 302 with a person's movement. The sensor 304 may be positioned to coordinate activation of the electrode 302 with each step taken by the user. The sensor 304 may be operably coupled to the foot portion 206 of the hollow sleeve 202. In the exemplary embodiment of FIG. 1, the sensor 304 is operably coupled to a heel portion 209 of the foot portion 206. Among other benefits, positioning the sensor 304 proximate to the heel portion 209 ensures that the sensor 304 contacts a ground surface while the user is walking or running. In some situations, the user's heel may experience a larger pressure while taking a step (e.g., while walking or running, etc.), making the force on the sensor 304 much easier to quantify. Additionally, placing the sensor 304 on the heel portion 209 provides a user with the ability to manually activate the electrode 302 (e.g., by tapping his/her heel on the ground or against their opposite heel, etc.). In other embodiments, the position of the sensor 304 may be different (e.g., on another position along the foot portion 206 of the hollow sleeve 202, etc.).

According to an exemplary embodiment, the sensor 304 is configured to generate a control signal in response to a force applied to the heel portion 209 of the hollow sleeve 202. The sensor 304 may include a pressure sensor. The pressure sensor may be a pressure-sensitive conductive sheet made from a material such as Velostat. The resistance of the conductive sheet may be a function of the pressure applied to the sheet or to the heel portion 209 of the hollow sleeve 202. The electrical impulse device 300 may be configured to activate the electrode 302 in response to the resistance of the conductive sheet dropping below a predetermined threshold. As shown in FIG. 1, the conductive sheet may extend throughout the heel portion 209 so as to maximize the contact area of the sheet, which can, advantageously, maximize the change in resistance resulting from leg movement.

In other embodiments, another form of textile pressure sensor or step detection may be utilized. In some embodiments, multiple sensors 304 may be utilized. Each sensor 304 may be disposed at a different location along the foot portion 206 of the hollow sleeve 202. Among other benefits, using multiple sensors 304 may improve detection of a specific type of user movement. For example, using multiple sensors 304 may help to identify movements that accompany the natural contraction of the calf muscle as compared to movements that do not result in contraction of the calf muscle. Coordinating the application of the electrical impulse with the natural contraction of the calf muscle can, advantageously, reduce pain associated with forced contraction (e.g., the contraction due to the electrical impulse).

As shown in FIG. 4, the electrical impulse device 300 includes a kinetic charger 316 and an indicator 318. Both the kinetic charger 316 and the indicator 318 are electrically coupled to the power source 308. The kinetic charger 316 is configured to supply a current to the power source 308 in response to user movement. According to an exemplary embodiment, the kinetic charger 316 is coupled to the hollow sleeve 202 (see also FIG. 1). The kinetic charger 316 may be coupled to the foot portion 206 of the hollow sleeve 202 so as to maximize the amount of energy transferred to the power source 308 during user movement (e.g., while walking, running, etc.). As with the electrode 302 and the power source 308, the kinetic charger 316 may be detachably coupled to the hollow sleeve 202 so that it may be easily replaced and/or removed from the hollow sleeve 202 when the sock 200 requires cleaning.

The indicator 318 is used to measure and report a condition of the electrical impulse device 300. In the exemplary embodiment of FIG. 4, the indicator 318 is configured to measure and report battery life for the electrical impulse device 300 (e.g., a charge level for the power source 308, etc.). The indicator 318 may include a light emitting diode (LED) that is configured to provide a visual indication of the remaining battery life to a user. The color of the LED may change as the battery is discharged. For example, the LED may be green when the battery is fully charged, yellow while the battery is discharging, and red when the battery needs to be charged or replaced. The indicator may be disposed on an outer surface of the hollow sleeve 202 so that it may be easily viewed by a user. In alternative embodiments, the indicator 318 may be a speaker configured to generate an alarm based on a determination that the remaining battery life is below a given threshold.

Various other conditions may be measured and reported by the indicator 318. For example, the indicator 318 may be configured to report an operating condition of the electrode 302 (e.g., whether the electrode 302 is activated, whether the electrode 302 is set to pulsate continuously, etc.) so as to provide a check for the user when diagnosing problems with the device 300. The indicator 318 may also be configured to signal the user to move after a predetermined period of inactivity is detected. The electrical impulse device 300 may include more or fewer indicators 318 depending on user requirements.

As shown in FIG. 4, the electrical impulse device 300 includes a step-counter 320 configured to monitor and record user movement. In an exemplary embodiment, the step-counter 320 is operably coupled to the hollow sleeve 202 (see also FIG. 1). As with other electrical components, the step-counter 320 may be detachable (e.g., removable) from the hollow sleeve 202 to facilitate replacement and/or to reduce the risk of damage to the step-counter 320 when the sock 200 is being washed. The step-counter 320 may include a pedometer or another form of movement tracking device. According to an exemplary embodiment, the step-counter 320 is configured to activate the electrode 320 after a predetermined period of time has elapsed during which the electrode is inactive. For example, the step-counter 320 may be configured to record each step that a user takes over time. The step-counter 320 may be configured to active the electrode 302 based on a determination that the number of steps is below a threshold number of steps during a given time interval (e.g., 0 steps over a 15 min period, etc.).

In some embodiments, sensor data from the step-counter 320 may be used to evaluate the effectiveness of the device during use. The sensor data could be compared with an amount of healing of a wound based on a clinical evaluation of a wound site. The data could be used to determine whether the device has been inactivated by a user, used improperly, etc. In some embodiments, the sensor data could be downloaded from the step-counter 320 using a computing device connected to the step-counter 320. In other embodiments, the device 300 may include a transceiver configured to wirelessly transmit sensor data (e.g., via Wi-Fi, Bluetooth, or another suitable wireless communication protocol). In some embodiments, the sensor data may be wirelessly transmitted to a personal fitness or health monitoring device such as a smart watch or smart phone (e.g., a mobile phone remotely connected to the device 300).

As shown in FIG. 4, the electrical circuit 500 includes a plurality of conductive members 306 to complete electrical connections between components. The electrical circuit 500 includes three circuits, shown as first circuit 502, second circuit 504, and third circuit 506. Alternatively, the electrical circuit 500 may include more or fewer circuits. As shown in FIG. 4, the first circuit 502 is configured, in part, to activate the electrode 302 whenever the sensor 304 detects user movement. The first circuit 502 includes the power source 308, the sensor 304, the step-counter 320, and the electrode 302 connected in series. The step-counter 320 interfaces the first circuit 502 between the sensor 304 and the electrode 302 so as to monitor and record operation of the electrode 302. The second circuit 504 includes the power source 308 and the indicator 318. The indicator 318 is connected across the power source 308 so as to monitor a charge level or a remaining capacity of the power source 308. The third circuit 506 includes the power source 308 and the kinetic charger 316, which actively recharges the power source 308 during normal use.

### Electrical Impulse Device Operation

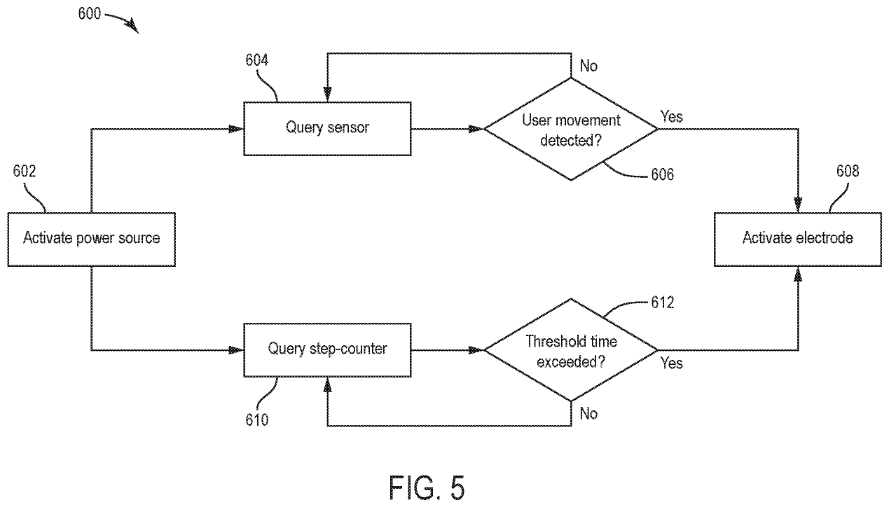

Referring now to FIG. 5, a method 600 of operating the electrical impulse device 300 (see also FIG. 4) is shown, according to an exemplary embodiment. The method 600 includes activating the power source 602 for the electrical impulse device 300. The power source 308 may be activated by connecting a battery to a conductive member 306 in the garment 100 (see also FIG. 1) or by actuating an on/off switch for the garment 100. In some embodiments, method 600 includes electrically connecting other components (e.g., the electrode 302, the sensor 304, the kinetic charger 316, the step-counter 320, etc.) to the conductive members 306 in the garment 100 and/or inserting one or more components into retaining pockets in the hollow sleeve 202 so as to position the components in the hollow sleeve 202.

The electrode 302 is configured to operate in either an activated state in which the electrode 302 is configured to stimulate the calf muscle or a deactivated state in which the electrode is not configured to stimulate the calf muscle. In the exemplary embodiment of FIG. 5, the method 600 includes using the sensor 304 and the step-counter 320 to control the operation of the electrode 302 (e.g., the operating state of the electrode 302, whether the electrode 302 is in the activated state or the deactivated state, etc.). As shown in FIG. 5, the method 600 includes querying the sensor 604 until user movement has been detected. The sensor 302 may be configured to generate a control signal in response to a pressure or force applied to the sensor 302. The control signal may include a switch that completes a circuit to power the electrode 302 in response to user movement.

As shown in FIG. 5, the method 600 includes detecting user movement 606 and activating the electrode 608 based on a determination that the user has taken a step. The electrode 302 may be configured to remain in the activated state until the pressure or force is removed from the sensor 302, thereby coordinating the electrical impulse with user movement (e.g., coordinating the impulse with a movement that results in contraction of the calf muscle, etc.). Among other benefits, coordinating the electrical impulse with user movement can reduce pain associated with an electrically induced contraction of the calf muscle and increase venous return as compared with an electrically induced contraction alone.

The method 600 includes operations that improve venous return while a user is at rest. The method 600 includes querying the step-counter 610 for any recorded user movements. The step-counter 320 (see also FIG. 4) may be configured to monitor and record a number of steps that the user has taken during a given time interval. The time interval may include a period of time up to and including a real-time (e.g., a current time, etc.). The time interval may be a time period of 10 min., 30 min., 1 hr., or another suitable time interval. The step-counter 320 may be configured to activate the electrode 608 based on a determination that a threshold time, during which the user has been immobile (e.g., during which no steps were taken), has been exceeded 612. Alternatively, the step-counter 320 may be configured to activate the electrode 608 based on a determination that a number of steps taken during the time interval is below a threshold number of steps.

According to an exemplary embodiment, the electrode 302 is configured to pulsate continuously between the activated state and the deactivated state after a predetermined period of time has elapsed during which the electrode 302 is in the deactivated state (e.g., during which no user movement has been detected). The duration between electrical impulses may vary depending on user preferences and treatment requirements.

### Making a Compression Garment for Stimulating Venous Return

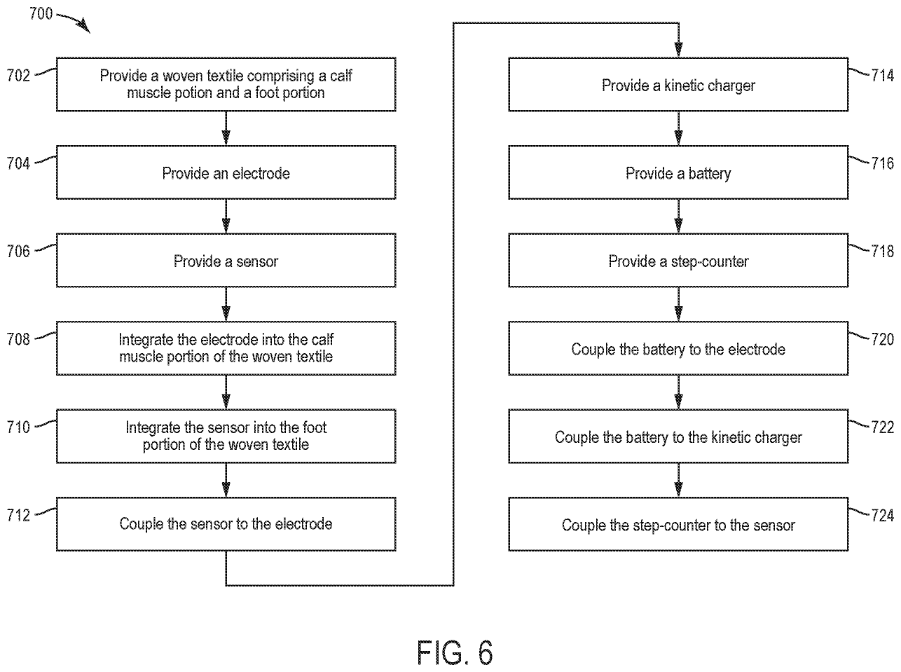

Referring now to FIG. 6, a method 700 of making a garment for stimulating venous return is shown, according to an exemplary embodiment. The method includes providing a woven textile including a calf muscle portion and a foot portion 702. The calf muscle portion may be configured to be disposed proximate to a calf muscle. The foot portion may be configured to be disposed proximate to a foot. The woven textile may be a sock or compression bandage configured to promote venous. The method 700 additionally includes providing an electrode 704 and providing a sensor 606. The electrode 704 may be configured to activate and stimulate the calf muscle in response to a control signal generated by the sensor.

The method 700 includes integrating the electrode into the calf muscle portion of the woven textile 708. The electrode may be inserted into a slot or pocket in the woven textile to secure the electrode in position relative to the woven textile. In some embodiments, a connector (e.g., an electrical connector) may be inserted into the electrode to operably couple the electrode to the woven textile. As shown in FIG. 6, the method 700 also includes integrating the sensor into the foot portion of the woven textile 710. As with the electrode integration operation, the operation of integrating the sensor may include inserting the sensor into a slot or pocket configured to retain the sensor in position with respect to the woven textile. The method 700 includes electrically coupling the sensor to the electrode 712. An electrical connector embedded in the woven textile may be inserted into the sensor to communicatively couple the sensor to the electrode.

As shown in FIG. 6, the method 700 includes providing additional electrical components that facilitate control and operation of the electrical impulse device. Operations include providing a kinetic charger 714 configured to supply a current in response to user movement, providing a battery 716 or other power source, providing a step-counter 718 configured to record a number of control signals, providing an indicator configured to measure and report battery life, etc. The method 700 may include integrating these components into the woven textile. As shown in FIG. 6, the method 700 additionally includes coupling (e.g., electrically connecting) the battery to at least one of the electrode 720 and the kinetic charger 722. The method 700 additionally includes communicatively coupling (e.g., electrically connecting) the step-counter to the sensor 724. In other exemplary embodiments, more or fewer operations may be performed to produce (e.g., make, manufacture, etc.) the garment.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A garment for stimulating venous return, comprising:
a woven textile comprising a calf muscle portion configured to be disposed proximate to a calf muscle, and a foot portion configured to be disposed proximate to a foot;
an electrode, the electrode operably coupled to the calf muscle portion; and
a sensor communicatively coupled to the electrode, the sensor disposed on the foot portion, the sensor configured to generate a control signal in response to a person's movement,
wherein the electrode is configured to activate the electrode in response to the control signal.

2. The garment of claim 1, wherein the foot portion of the woven textile comprises a heel portion configured to be disposed proximate to a heel of the foot, wherein the sensor is operably coupled to the heel portion, the sensor comprising a pressure sensor configured to generate the control signal in response to a force applied to the heel portion, and wherein the pressure sensor is configured to deactivate the control signal in response to the force being removed from the heel portion.

3. The garment of claim 1, wherein the calf muscle portion of the woven textile comprises a common perineal nerve portion configured to be disposed proximate to a common perineal nerve, and wherein the electrode is disposed proximate to the common perineal nerve portion.

4. The garment of claim 1, wherein the electrode is configured to operate in either an activated state in which the electrode is configured to stimulate the calf muscle or a deactivated state in which the electrode is not configured to stimulate the calf muscle, and wherein the electrode is configured to pulsate continuously between the activated state and the deactivated state after a predetermined period of time has elapsed during which the electrode is in the deactivated state.

5. The garment of claim 1, further comprising a kinetic charger and a battery, wherein the kinetic charger is electrically coupled to the battery, wherein the battery is electrically coupled to the electrode, and wherein the kinetic charger is configured to supply a current to the battery in response to the person's movement.

6. The garment of claim 1, further comprising conductive fibers woven into the woven textile and electrically coupled to at least one of the electrode and the sensor.

7. An assembly for stimulating venous return, comprising:
a sock comprising a hollow sleeve, the hollow sleeve comprising a calf muscle portion configured to receive a calf muscle and a foot portion configured to receive a foot, the hollow sleeve configured to apply a pressure to the calf muscle and the foot;
an electrode disposed in the hollow sleeve proximate to the calf muscle portion; and
a sensor disposed in the hollow sleeve proximate to the foot portion, the sensor communicatively coupled to the electrode, the sensor configured to generate a control signal in response to a person's movement,
wherein the electrode is configured to activate the electrode in response to the control signal.

8. The assembly of claim 7, wherein the foot portion further comprises a heel portion configured to be disposed proximate to a heel of the foot, and wherein the sensor is disposed proximate to the heel portion, the sensor comprising a pressure sensor configured to generate the control signal in response to a presence or absence of a force applied to the heel portion.

9. The assembly of claim 7, wherein the calf muscle portion of the hollow sleeve comprises a common perineal nerve portion configured to be disposed proximate to a common perineal nerve, and wherein the electrode is disposed proximate to the common perineal nerve portion.

10. The assembly of claim 7, wherein the electrode is configured to operate in either an activated state in which the electrode activates to stimulate the calf muscle or a deactivated state in which the electrode deactivates and does not stimulate the calf muscle, and wherein the electrode is configured to pulsate continuously between the activated state and the deactivated state after a predetermined period of time has elapsed during which the electrode is in the deactivated state.

11. The assembly of claim 7, further comprising a step-counter disposed in the hollow sleeve, the step-counter configured to record the person's movement.

12. The assembly of claim 11, the step-counter configured to record a number of control signals generated by the sensor.

13. The assembly of claim 7, wherein the sock comprises a long stretch material.

14. The assembly of claim 7, further comprising conductive fibers woven into the sock and electrically coupled to at least one of the electrode and the sensor.

15. The assembly of claim 7, wherein at least one of the electrode and the sensor are removably coupled to the sock.

16. The assembly of claim 7, wherein at least one of the electrode and the sensor are woven into the sock.

17. The assembly of claim 7, further comprising a kinetic charger disposed in the hollow sleeve and a battery disposed in the hollow sleeve, wherein the kinetic charger is electrically coupled to the battery, wherein the battery is electrically coupled to the electrode, and wherein the kinetic charger is configured to supply a current to the battery in response to the person's movement.

18. A method of making a compression garment for stimulating venous return, comprising:
providing a woven textile comprising a calf muscle portion configured to be disposed proximate to a calf muscle, and a foot portion configured to be disposed proximate to a foot;
providing an electrode, the electrode configured to activate and stimulate the calf muscle in response to a control signal;
providing a sensor, the sensor configured to generate the control signal in response to an applied force;
integrating the electrode into the calf muscle portion of the woven textile;
integrating the sensor into the foot portion of the woven textile; and
electrically coupling the sensor to the electrode.

19. The method of claim 18, further comprising:
providing a kinetic charger configured to supply a current in response to a person's movement;
providing a battery;
electrically coupling the battery to the kinetic charger; and
electrically coupling the battery to the electrode.

20. The method of claim 18, further comprising:
providing a step-counter configured to record a number of control signals; and
electrically coupling the step-counter to the sensor.
